(19) United States
(12) Patent Application Publication
(10) Pub. No.: US 2025/0232435 A1
(43) Pub. Date: Jul. 17, 2025

(54) INFORMATION PROCESSING DEVICE, INFORMATION PROCESSING METHOD, AND PROGRAM

(71) Applicant: SONY GROUP CORPORATION, TOKYO (JP)

(72) Inventors: MINORI MORITA, TOKYO (JP); HIROYASU BABA, TOKYO (JP); TAKANORI FUKAZAWA, TOKYO (JP); NANA KAWAMURA, TOKYO (JP)

(21) Appl. No.: 18/730,735

(22) PCT Filed: Jan. 12, 2023
(86) PCT No.: PCT/JP2023/000540

§ 371 Date: Jul. 19, 2024

(30) Foreign Application Priority Data
Jan. 27, 2022 (JP) 2022-011051

(51) Int. Cl. G06T 7/00; A61B 5/00; G06T 7/90; G06V 30/19; G16H 10/60; G16H 30/20; H04N 5/76

(52) U.S. Cl. CPC G06T 7/0012; A61B 5/0077; A61B 5/7282; G06T 7/90; G06V 30/19; G16H 10/60; G16H 30/20; H04N 5/76; A61B 2576/02; G06T 2207/10016; G06T 2207/30004; G06T 2207/30201; G06T 2207/30232

(57) ABSTRACT

The present technology relates to an information processing device, an information processing method, and a program that enable appropriate monitoring of a condition of a patient even from a remote location. There are provided an acquisition unit that acquires at least video data from an imaging device that images a patient and video data from an imaging device that images an instrument monitoring the patient; and a detection unit that detects an abnormality of a condition of the patient by analyzing the video data acquired by the acquisition unit. A plurality of imaging devices that images a plurality of instruments monitoring the patient is grouped by reading a patient ID assigned to the patient using the imaging device. The present technology can be applied to an information processing device that monitors the condition of the patient.

### TECHNICAL FIELD

The present technology relates to an information processing device, an information processing method, and a program, and for example to an information processing device, an information processing method, and a program capable of monitoring a state of a patient even at a distant place.

### BACKGROUND ART

In the related art, for example, in a hospital provided with an intensive care unit (ICU), a medical worker performs monitoring work of monitoring a status of a patient admitted to the ICU and recording the monitored content electronically or on paper. In order to perform such monitoring work, the medical worker needs to go to a hospital room and check the status of the patient, and also spends a lot of time on the monitoring work, so that the efficiency of the monitoring work is required.

Patent Document 1 proposes a nurse call system that enables a medical worker to remotely check the status of the patient using a hospital room camera without visiting the hospital room.

### CITATION LIST

### Patent Document

Patent Document 1: Japanese Patent Application Laid-Open No. 2019-162241

### SUMMARY OF THE INVENTION

### Problems to be Solved by the Invention

In Patent Document 1, the status of the patient is checked using the hospital room camera. However, since information cannot be obtained from instruments connected to the patient, such as a vital instrument, a drainage drain, and a drip syringe, the medical worker needs to go to the hospital room in order to obtain information from these instruments.

It is desired to be able to check a condition of a patient including the instrument for sensing the condition of the patient and to improve the efficiency of the monitoring work.

The present technology has been made in view of such a situation, and makes it possible to acquire information from a plurality of instruments even at a remote location and to perform processing using the acquired information.

### Solutions to Problems

An information processing device according to an aspect of the present technology is an information processing device including an acquisition unit that acquires at least video data from an imaging device that images a patient and video data from an imaging device that images an instrument monitoring the patient; and a detection unit that detects an abnormality of a condition of the patient by analyzing the video data acquired by the acquisition unit.

An information processing method according to an aspect of the present technology is an information processing method including, by an information processing device, acquiring at least video data from an imaging device that images a patient and video data from an imaging device that images an instrument monitoring the patient; and detecting an abnormality of a condition of the patient by analyzing the acquired video data.

A program according to an aspect of the present technology is a program for causing a computer that controls an information processing device to execute processing including steps of: acquiring at least video data from an imaging device that images a patient and video data from an imaging device that images an instrument monitoring the patient; and detecting an abnormality of a condition of the patient by analyzing the acquired video data.

In the information processing device, the information processing method, and the program according to the aspects of the present technology, at least the video data from the imaging device that images the patient and the video data from the imaging device that images the instrument monitoring the patient are acquired, and the abnormality of the condition of the patient is detected by analyzing the acquired video data.

Note that the information processing device may be an independent device or an internal block constituting one device.

Note that a program can be provided by being transmitted via a transmission medium or being recorded on a recording medium.

### BRIEF DESCRIPTION OF DRAWINGS

FIG. 1 is a diagram illustrating a configuration of an embodiment of an information processing system to which the present technology is applied.

FIG. 2 is a diagram for describing an installation method of a camera.

FIG. 3 is a diagram for describing an installation method of a camera that images a plurality of instruments.

FIG. 4 is a diagram illustrating a configuration example of a server.

FIG. 5 is a diagram illustrating a functional configuration example of the server.

FIG. 6 is a diagram for describing an operation of the information processing system.

FIG. 7 is a diagram for describing an operation of the server.

FIG. 8 is a diagram for describing a method of estimating a facial expression.

FIG. 9 is a diagram for describing a method of estimating a posture.

FIG. 10 is a diagram illustrating a screen example displayed on a display unit.

FIG. 11 is a diagram illustrating a screen example displayed on the display unit.

FIG. 12 is a diagram illustrating a screen example displayed on the display unit.

### MODE FOR CARRYING OUT THE INVENTION

Hereinafter, a mode for carrying out the present technology (hereinafter, referred to as an embodiment) will be described.

### <Information Processing System>

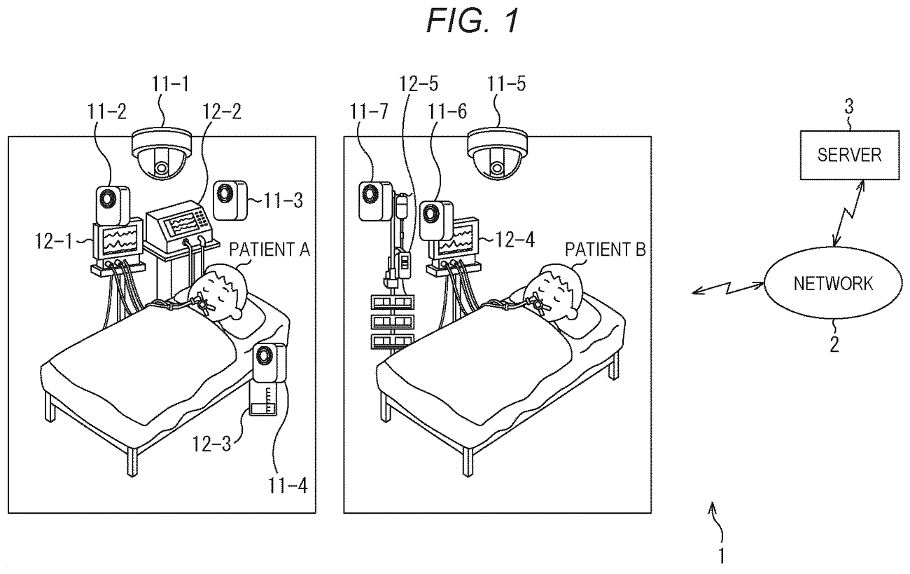

FIG. 1 is a diagram illustrating a configuration of an embodiment of an information processing system to which the present technology is applied. Since the information processing system to which the present technology is applied can be applied to, for example, a system for monitoring a patient, the description will be continued here using a system for monitoring a patient as an example.

An information processing system 1 that monitors a patient includes a plurality of cameras and instruments as the sensing target, and is a system that performs abnormality detection and sudden change prediction of the patient by analyzing an image captured by the cameras. Information on the abnormality detection and sudden change prediction is provided to a medical worker. The medical worker includes a doctor and a nurse. In the present specification, the system represents the entire device including a plurality of devices.

Cameras 11-1 to 11-4 and instruments 12-1 to 21-3 are installed on the bedside of a patient A. Cameras 11-5 to 11-7 and instruments 12-4 and 21-5 are installed on the bedside of a patient B. The cameras 11-1 to 11-7 are configured to be able to exchange data with a server 3 via a network 2. Hereinafter, in a case where it is not necessary to individually distinguish the cameras 11-1 to 11-7, the cameras are simply referred to as the camera 11. The same applies to other parts.

The network 2 is a wired or/and wireless network, and may include a local area network (LAN) or the Internet. As will be described later, in a case where there is a relationship between a master unit and a slave unit between the cameras 11, communication is also performed between the master unit and the slave unit.

The camera 11-1 is an imaging device that images the patient A as a subject, and is an imaging device installed at a position where the face, the entire body, the operated part, and the like of the patient A can be imaged. The camera 11-1 functions as a camera for acquiring video data for detecting the complexion, facial expression, posture, and the like of the patient A.

The camera 11-2 is an imaging device that images the instrument 12-1 as a subject, and the instrument 12-1 is, for example, an instrument that measures and displays a vital sign. The camera 11-2 functions as, for example, a camera for vital signs, and is an imaging device installed at a position where a screen of the instrument 12-1 can be imaged.

The camera 11-3 is an imaging device that images the instrument 12-2 as a subject, and the instrument 12-2 is, for example, a ventilator. The camera 11-3 functions as a camera for a respirator, and is an imaging device installed at a position where a screen of the instrument 12-2 can be imaged.

The camera 11-4 is an imaging device that images the instrument 12-3 as a subject, and the instrument 12-3 is, for example, a drainage drain. The camera 11-4 is an imaging device installed at a position where the color and amount of the drainage in the drainage drain as the instrument 12-3 can be imaged.

The camera 11-5 is an imaging device that images the patient B as a subject, and is an imaging device installed at a position where the face, the entire body, the operated part, and the like of the patient B can be imaged. The camera 11-1 functions as a camera for acquiring video data for detecting the complexion, facial expression, posture, and the like of the patient B.

The camera 11-6 is an imaging device that images the instrument 12-4 as a subject, and the instrument 12-4 is, for example, an instrument that measures and displays a vital sign. The camera 11-7 functions as, for example, a camera for vital signs, and is an imaging device installed at a position where a screen of the instrument 12-4 can be imaged.

The camera 11-7 is an imaging device that images the instrument 12-5 as a subject, and the instrument 12-5 is, for example, a drip syringe. The camera 11-7 functions as a camera for a drip syringe, and is an imaging device installed at a position where the remaining amount of the drip of the drip syringe as the instrument 12-5 can be imaged.

The camera 11 may be a camera that can perform zooming, panning, tilting, and the like by remote control. For example, in a case where the face of the patient is imaged, the face may be enlarged and imaged using a zoom function.

The camera 11 functions as an imaging device that images an instrument installed in a medical site, a patient, or the like as a subject. The instrument 12 functions as a monitoring device that monitors a patient. In a case where the camera 11 is imaging a patient, the camera 11 itself functions as a monitoring device for monitoring the patient.

The video of the subject imaged by the camera 11 is supplied to the server 3 via the network 2.

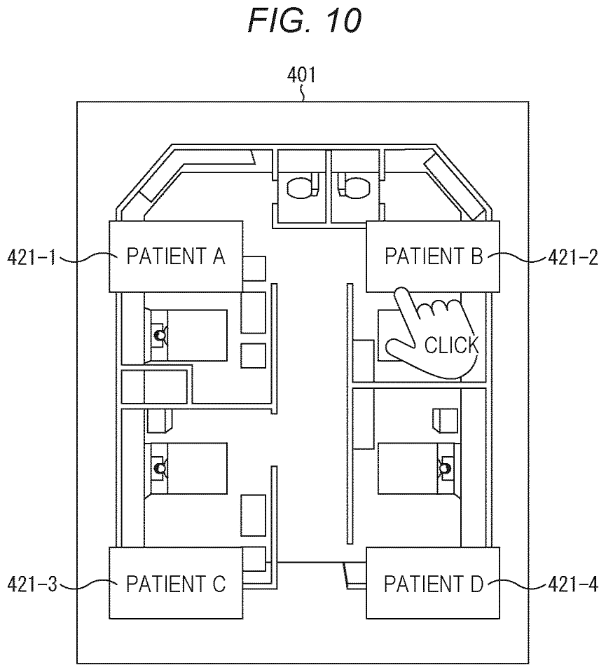

The server 3 can include, for example, a personal computer (PC), performs abnormality detection and sudden change prediction of a patient using data acquired from the camera 11, displays information associated with information regarding the detected condition on a display unit 401 (FIG. 10), and provides the information to a medical worker.

As the camera 11, for example, an RGB camera, a depth camera, an infrared (IR) camera, or the like can be used. Since the depth camera can more accurately estimate the posture of a person in a case where the person is imaged as a subject, the depth camera can be applied to the camera 11 that images the person. Since the IR camera is suitable for acquiring a video at night (dark place), the IR camera can be applied to the camera 11 that images the instrument 12 that needs to be imaged at night.

In the description with reference to FIG. 1, the case where the instrument 12 is imaged by the camera 11 and the information obtained from the instrument 12 is supplied to the server 3 via the camera 11 has been described as an example. However, as long as the instrument can directly supply the information from the instrument 12 to the server 3, the information may be directly supplied from the instrument 12 to the server 3.

That is, the information processing system 1 may have a configuration in which the instrument 12 that directly supplies information from the instrument 12 to the server 3 and the instrument 12 that indirectly supplies information to the server 3 via the camera 11 are mixed. In the following description, a case where only the instrument 12 that indirectly supplies information to the server 3 via the camera 11 is configured will be described as an example.

In the following description, for simplification of description, it is assumed that a patient is also included in the instrument 12. For example, the description that the instrument 12 is imaged by the camera 11 includes a case where the instrument 12 that measures vital signs is imaged by the camera 11 and a case where a patient is imaged by the camera 11.

### <Method of Fixing Camera>

As described above, the camera 11 images the instrument 12. For example, in a case where the instrument 12-1 is an instrument that measures and displays a vital sign, the camera 11-3 that images the instrument 12-1 is installed at a position where the displayed vital sign is appropriately imaged.

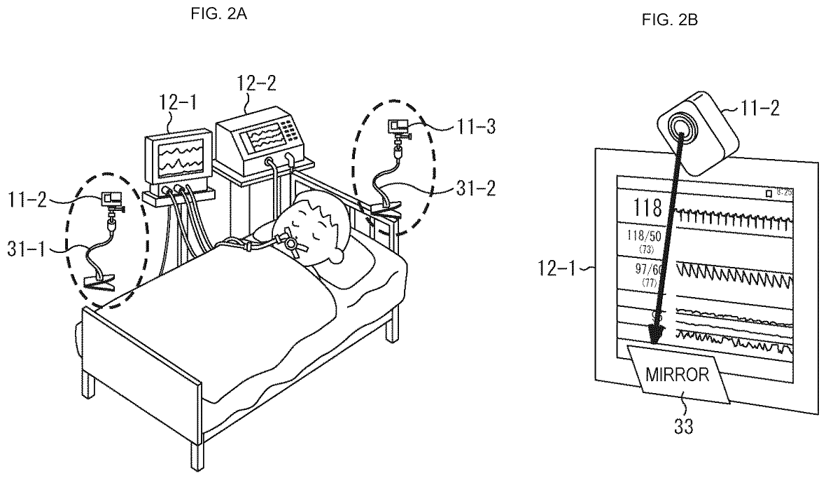

For example, as illustrated in A of FIG. 2, an arm 31-1 is attached to a frame of a bed, the camera 11-2 is attached to the arm 31-1, and thereby the camera 11-2 is fixed to the bed. Furthermore, the camera 11-2 is fixed by being oriented in a direction in which the instrument 12-1 falls within the angle of view.

Similarly, an arm 31-2 is attached to the frame of the bed, the camera 11-3 is attached to the arm 31-2, and thereby the camera 11-3 is fixed to the bed. Furthermore, the camera 11-3 is fixed by being oriented in a direction in which the instrument 12-2 falls within the angle of view. The other cameras 11 are also fixed at positions where the instrument 12 in charge can be imaged, using the arms 31.

Note that the attachment position of the arm 31 may be other than the frame of the bed. In a case where the camera 11 itself is provided with a clip or the like and has a structure in which the camera 11 can be fixed to the frame of the bed or the like without using the arm 31, the camera 11 may be fixed at a position where the instrument 12 in charge can be imaged, using the structure.

As illustrated in B of FIG. 2, a mechanism may be provided in which a mirror 33 is attached to the instrument 12 and an image reflected in the mirror 33 is imaged by the camera 11-2. In the example illustrated in B of FIG. 2, the mirror 33 is fixed to the lower side of the instrument 12-1. The camera 11-2 is fixed to the upper side of the instrument 12-1. The camera 11-2 and the mirror 33 are fixed at positions where the camera 11-2 can image the screen of the instrument 12-1 reflected in the mirror 33 is captured.

In this manner, a mechanism in which the camera 11 is fixed to the instrument 12 may be provided using the mirror 33.

For example, since many instruments 12 are arranged around the bed in an intensive care unit (ICU), by arranging the cameras 11 as described above, the cameras 11 can be arranged so as not to interfere with the arranged instruments 12, and it is possible to arrange the cameras 11 with the operating line of the medical worker secured.

In the following description, basically, a case where one camera 11 is associated with one instrument 12 will be described as an example, but one camera 11 may image a plurality of instruments 12, in other words, one camera 11 may be associated with a plurality of instruments 12.

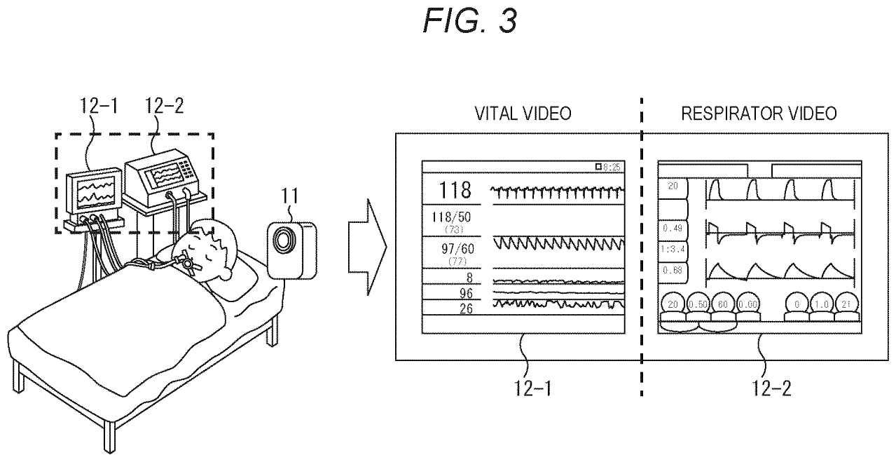

For example, as illustrated in FIG. 3, one camera 11 images two instruments 12, that is, the instrument 12-1 and the instrument 12-2. As illustrated in the right diagram of FIG. 3, the screen of the video imaged by the camera 11 is such that the screens of the instrument 12-1 and the instrument 12-2 are projected to the left and right. For example, in a case where the instrument 12-1 is an instrument that measures vital signs and the instrument 12-2 is an instrument that measures respiration, a video in which a vital video and a respirator video are displayed on one screen is imaged by the camera 11.

Although details will be described later, in a case where the server 3 acquires a video in which the two instruments 12 are imaged as illustrated in the right diagram of FIG. 3, the server 3 divides the screen and analyzes each of the vital video and the respirator video.

As described above, in a case where a plurality of instruments 12 is imaged by one camera 11, the number of cameras 11 to be installed can be reduced, and the storage capacity on the server 3 side for storing the video from the camera 11 can also be reduced.

### <Configuration of Server>

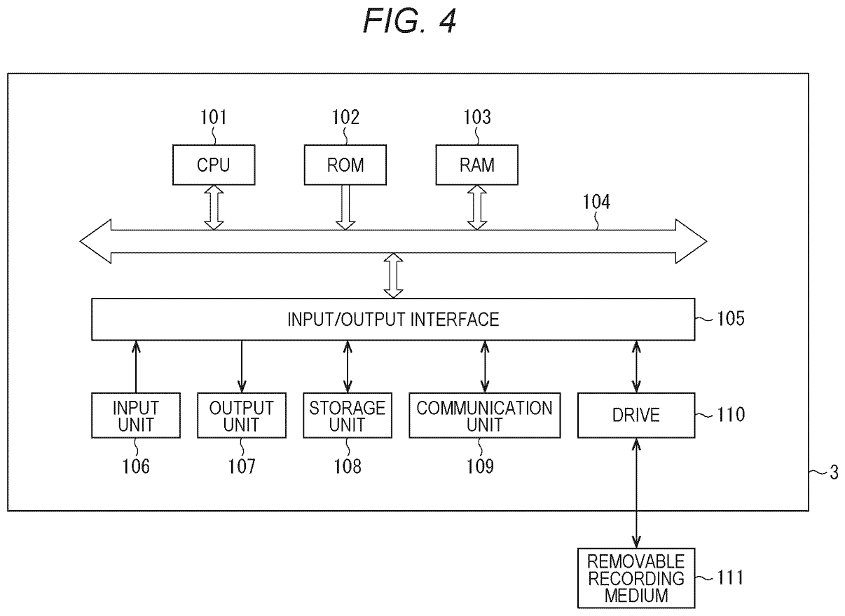

FIG. 4 is a block diagram illustrating a configuration example of the server 3. In the server 3, a central processing unit (CPU) 101, a read only memory (ROM) 102, and a random access memory (RAM) 103 are mutually connected by a bus 104. Moreover, an input/output interface 105 is connected to the bus 104. An input unit 106, an output unit 107, a storage unit 108, a communication unit 109, and a drive 110 are connected to the input/output interface 105.

The input unit 106 includes a keyboard, a mouse, a microphone, and the like. The output unit 107 includes a display, a speaker, and the like. The storage unit 108 includes a hard disk, a non-volatile memory, and the like. The communication unit 109 includes a network interface, and the like. The drive 110 drives a removable medium 111 such as a magnetic disk, an optical disk, a magneto-optical disk, or a semiconductor memory.

In a computer configured as described above, the CPU 101 loads, for example, a program stored in the storage unit 108 into the RAM 103 via the input/output interface 105 and the bus 104, and executes the program to thereby perform the series of processing described below.

The program executed by the server 3 (CPU 101) can be provided by being recorded in the removable medium 111 as a package medium or the like, for example. Furthermore, the program can be provided via a wired or wireless transmission medium such as a local area network, the Internet, or digital satellite broadcasting.

In the server 3, the program can be installed in the storage unit 108 via the input/output interface 105 by attaching the removable medium 111 to the drive 110. Furthermore, the program can be received by the communication unit 109 via the wired or wireless transmission medium and installed on the storage unit 108. Furthermore, the program can be installed on the ROM 102 or the storage unit 108 in advance.

Note that, a program to be executed by the computer may be a program by which processing is performed in time series in the order described in the present specification, or may be a program by which processing is performed in parallel or at a required time such as when a call is made.

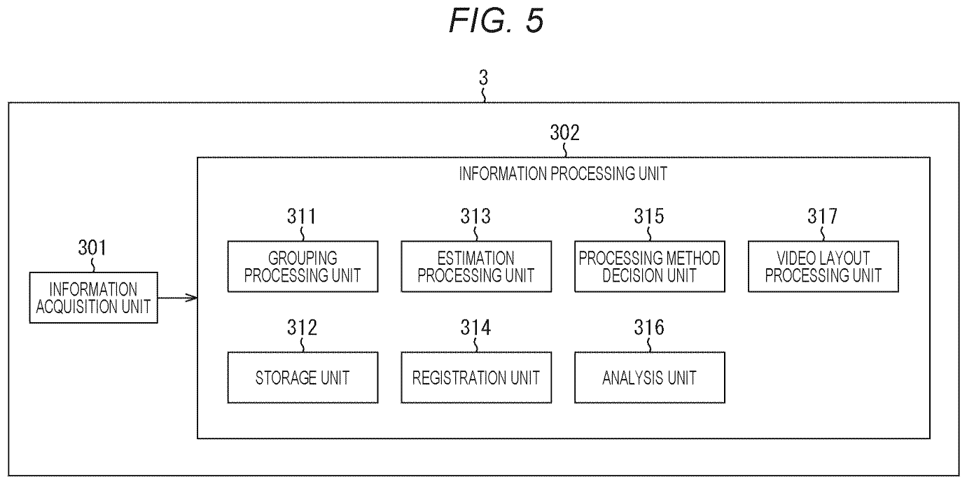

FIG. 5 illustrates functional blocks of the server 3. For example, as described above, the CPU 101 loads a program stored in the storage unit 108 into the RAM 103 and executes the program, so that some or all of the functions are realized.

The server 3 includes an information acquisition unit 301 and an information processing unit 302. The information acquisition unit 301 acquires information from the camera 11 via the network 2 (FIG. 1). The information acquired by the information acquisition unit 301 is supplied to the information processing unit 302. The information processing unit 302 processes the supplied information.

The information processing unit 302 includes a grouping processing unit 311, a storage unit 312, an estimation processing unit 313, a registration unit 314, a processing method decision unit 315, an analysis unit 316, and a video layout processing unit 317.

### <Processing in Server>

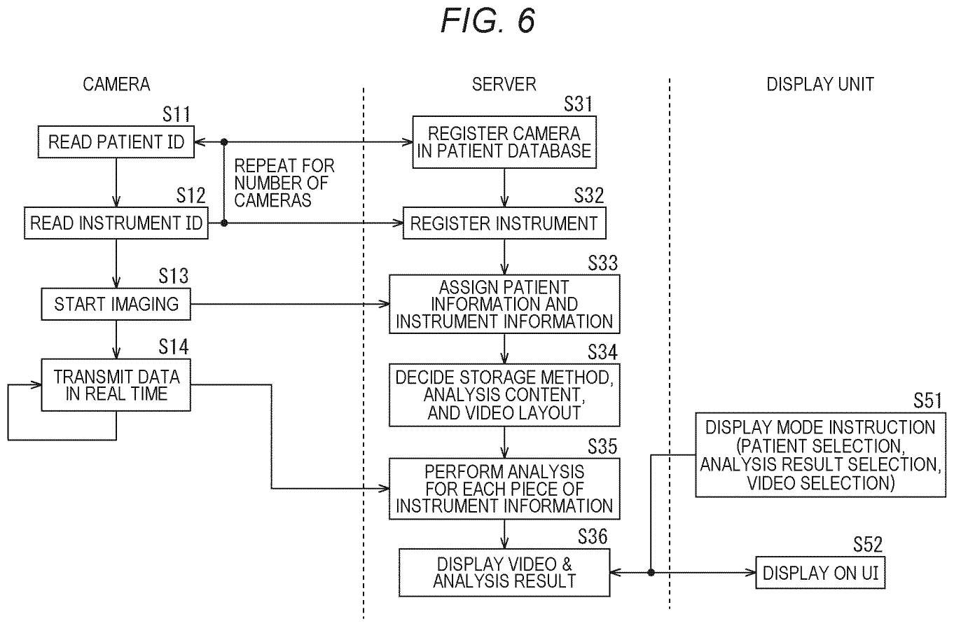

The processing performed in the information processing system 1 and the processing performed in each unit of the server 3 will be additionally described with reference to the flowchart of FIG. 6.

The processing of the information processing system 1 is roughly divided into grouping processing of grouping and registering a plurality of cameras 11 associated with a predetermined patient and/or bed and giving a group identifier. There is estimation processing of estimating the instrument 12 to be imaged by each of the grouped cameras 11.

As a result of the estimation processing, in a case where it is specified what the instrument 12 is sensing, there is decision processing of deciding an analysis method and a data storage method according to the specified sensing. There is storage processing for the storage in the storage unit 312 on the basis of the storage method decided by the decision processing.

There is display processing of deciding a layout in a user interface (UI) including the analysis result based on the data from the plurality of cameras 11 and displaying the UI based on the decided layout. These kinds of processing will be described.

### <Grouping Processing>

Processing related to the grouping processing is executed on each of the camera 11 side and the server 3 side. In step S11, the camera 11 reads a patient ID. The patient ID is, for example, an ID described on a name plate affixed to the bed or described on a wristband affixed to an arm or a foot of the patient, and is a number, a character, a symbol, a character string including a combination thereof, or the like assigned to uniquely specify the patient.

For example, the medical worker operates the camera 11 and images the patient ID described in the wristband, and thereby the patient ID is read. The patient ID read by the camera 11 is supplied to the server 3.

In step S31, the server 3 acquires the patient ID supplied from the camera 11 using the information acquisition unit 301, and supplies the patient ID to the grouping processing unit 311 of the information processing unit 302. The grouping processing unit 311 of the server 3 searches the storage unit 312 on the basis of the supplied patient ID, and registers the camera 11 in association with the patient.

The server 3 holds a patient database. The patient database can be constructed in the storage unit 312 (FIG. 5). Here, the description will be continued on the assumption that the patient database is constructed in the storage unit 312. However, the patient database may be constructed separately from the server 3, and the server 3 may access and refer to the patient database as necessary.

The server 3 registers the camera 11 in the patient database in association with the patient ID (patient). A medical record may be stored in the patient database, and information associated with information regarding the camera 11 may be registered in the medical record.

Such processing of registering the camera 11 (referred to as camera registration method 1) is performed for the number of cameras 11 desired to be associated with a predetermined patient and/or a predetermined bed. A patient to be observed and a plurality of cameras 11 installed for observing a vital sign or the like of the patient are registered in association with each other. That is, the plurality of cameras 11 installed for observing the condition of a predetermined patient are grouped.

A grouping identifier is assigned to the grouped cameras 11 so that it can be identified from a plurality of cameras 11 observing other patients. The grouping identifier may be a patient ID.

As a camera registration method 2, the plurality of cameras 11 may be divided into a master unit and a slave unit, and the master unit may be registered in the server 3 to perform grouping of the plurality of cameras 11. One of the plurality of cameras 11 is set as the master unit, and the remaining cameras 11 are set as the slave units. For example, in the patient A in FIG. 1, the camera 11-1 is set as the master unit, and the cameras 11-2 to 11-4 are set as the slave units. The slave units are registered in the master unit.

The master unit and the slave unit are paired by, for example, Wi-Fi, Bluetooth (registered trademark), near field communication (NFC), or the like. In such a case, the master unit is registered in the server 3, so that the cameras 11 associated with a predetermined patient can be registered. In this case, for example, the patient ID described in the wristband is read by the master unit, and the read patient ID is supplied to the server 3 side and registered.

As a camera registration method 3, similarly to the camera registration method 2, one of the plurality of cameras 11 is set as the master unit, and the other cameras 11 are set as the slave units. The face of the patient is imaged by the camera 11 serving as the master unit, and data of the face image of the patient is supplied to the server 3 side as the patient ID. In the server 3, the camera 11 is registered by collating with face image data of a patient registered in advance, specifying the patient, and associating the specified patient with the camera 11.

The camera registration method 3 can also be applied to the camera registration method 1, and the face of the patient may be imaged by each camera 11 to be registered, and the face image data may be supplied to the server 3 side as the patient ID.

As a camera registration method 4, a plurality of cameras 11 may be grouped and set in advance, and may be registered in the server 3. For example, five cameras 11 are registered as one group, and a grouping identifier allocated to the five cameras 11 and a patient are registered in association with each other on the server 3 side.

As a camera registration method 5, cameras 11 of which the distance is close to each other are grouped, and a grouping identifier is assigned. In a case where the distance between the cameras 11 is short, there is a high possibility that the cameras 11 are installed near a predetermined bed, and the cameras 11 observing a predetermined patient can be grouped by grouping such cameras 11. Whether or not the distance between the cameras 11 is short can be determined by using the Wi-Fi strength, and the cameras 11 having the same Wi-Fi strength can be determined to be close to each other and grouped.

As a camera registration method 6, an indoor camera that images the entire room including the patient and the bed is provided, the camera 11 installed around the bed (around the patient) is detected by the indoor camera, the plurality of detected cameras 11 is grouped, and a grouping identifier is assigned.

The grouping of the cameras 11 is performed by any one of or a combination of the camera registration methods 1 to 6. In a case where there is an instrument 12 that supplies the data to the server 3 without passing through the camera 11, the instrument is grouped to be included in the same group as the cameras 11 by a method different from the above-described registration method.

In a case where a medical worker notices that an erroneous camera 11 has been grouped after performing processing for grouping the cameras 11, a mechanism capable of releasing the grouping is provided. For example, a cancel barcode for canceling the registration is prepared, and the camera 11 reads the cancel barcode to release the grouping.

At the time when another patient ID is read by the camera 11 of which the grouping is desired to be released, the grouping of the camera 11 may be released, and the camera may be grouped into a group managed by the newly read another patient ID.

A mechanism may be provided in which a recognition sound that causes the medical worker to recognize that the camera 11 is associated with (registered) the patient or recognize that the grouping has been released is output at the time of registration or release. The recognition sound may be a beep sound or a certain message. For example, messages such as “ . . . has been registered in the patient ID . . . ” and “ . . . has been released in the patient ID . . . ” may be output.

The recognition sound may be output from the camera 11 or may be output from the output unit 107 (FIG. 4) on the server 3 side.

There is a possibility that the cameras 11 grouped by applying any one of the camera registration methods 1 to 6 described above are disconnected from the server 3 due to a wireless state or the like. Furthermore, there is also a possibility that the connection between the master unit and the slave unit is disconnected. In a case where the connection between the camera 11 and the server 3 is disconnected, an alert to notify the medical worker of the disconnection may be issued.

For example, a message, a mark, or the like that causes the medical worker to recognize that the connection has been lost may be displayed on the display unit 401 (FIG. 10) as the output unit 107 of the server 3. When this display is made, a mechanism may be provided in which a candidate determined to be disconnected from the strength of the radio wave is picked up and presented to the medical worker, and the medical worker can select the candidate. Furthermore, in a case where the selection is made, reconnection with the selected camera 11 may be performed.

In a case where the connection is disconnected, the registration may be performed again by the medical worker by the method applied among the camera registration methods 1 to 6 described above. In a case where the camera 11 of another master unit or slave unit shows the camera 11 of which the connection is determined to be disconnected, a function of automatically performing association again without bothering the medical worker may be provided.

Such processing related to the registration of the camera 11 and the instrument 12 is performed by the registration unit 314.

### <Estimation Processing>

In a case where the grouping processing is performed in this manner, the estimation processing starts. The estimation processing is processing of estimating what is set as the sensing target by the instrument 12 that is being imaged by the camera 11.

In step S12, the camera 11 reads an instrument ID. The instrument ID is, for example, a number, a barcode, a QR code (registered trademark), or the like assigned to the instrument 12, and a seal on which a number, a barcode, a QR code, or the like is printed is affixed to a side surface or the like of the instrument 12. The camera 11 reads the instrument ID by reading the number printed on the affixed seal. By reading the instrument ID, it is possible to specify what type of sensing the instrument 12 performs.

In step S32, the server 3 registers the instrument 12. What kind of instrument 12 the camera 11 that has supplied the patient ID is imaging is registered. For this purpose, the instrument ID read from the instrument 12 being imaged is supplied from the camera 11 side. For example, a table in which the instrument ID and the measurement target are associated with each other is held in the storage unit 312 such that what kind of vital sign the instrument 12 measures is specified by the instrument ID. Alternatively, a mechanism for searching the Internet from the instrument ID to specify the measurement target may be provided (referred to as estimation method 1).

As another estimation method (referred to as estimation method 2), a method of imaging the instrument 12 using the camera 11 and collating from an instrument database registered in advance may be applied. In step S12, the camera 11 images the instrument 12, and supplies image data of the instrument 12 to the server 3 as the instrument ID. In step S32, the server 3 refers to, for example, the instrument database stored in the storage unit 312, specifies an instrument matching the instrument 12, and reads information.

The instrument database is a database in which image data of the instrument 12 and a function, for example, a function such as what vital sign is measured are associated with each other. The instrument database may be provided in a place other than the server 3 and referred to as necessary. In such a configuration, it is also possible to access the instrument database via the Internet to perform the search.

In a case where the instrument 12 imaged by the camera 11 is imaging a urinary bladder, it is estimated that the instrument 12 is a urine drainage bag and the camera 11 is a camera imaging (sensing) the urine drainage bag. In a case where the instrument 12 imaged by the camera 11 is the face of the patient, it is estimated that the instrument 12 is the face of the patient and the camera 11 is a camera imaging the face of the patient. In a case where the instrument 12 imaged by the camera 11 is imaging the drip syringe, it is estimated that the instrument 12 is the drip syringe and the camera 11 is a camera imaging (sensing) the drip syringe.

As still another estimation method (referred to as estimation method 3), the vital signs displayed on the display unit of the instrument 12 imaged by the camera 11 may be read to estimate the sensing target of the instrument 12. Furthermore, in a case where the instrument 12 is an instrument that directly supplies data to the server 3, the type of vital sign sensed by the instrument 12 may be estimated by analyzing metadata from the instrument 12.

In a case where the estimation of the instrument 12 is wrong, a mechanism for correcting the wrong estimation is provided. For example, the estimation result displayed on the display unit 401 (FIG. 10) as the output unit 107 of the server 3 is checked by the medical worker, and is corrected in a case where there is an error.

As in the estimation method 2 described above, a function of preventing erroneous estimation may be provided by reading a barcode or the like affixed to the instrument 12 using the camera 11 and collating the function of the instrument 12 based on the read barcode or the like with the estimated function.

In a case where a wrong instrument 12 is erroneously registered, a mechanism for canceling the registration is provided. For example, the registration of the instrument 12 imaged by the camera 11 is released by causing the camera 11 to read a cancel barcode for canceling the registration.

At the time when the instrument 12 of which the registration is to be canceled starts being imaged by another camera 11, the registration of the instrument 12 may be canceled, and the instrument 12 may be registered in association with the other camera 11 that has newly started imaging.

A mechanism may be provided in which a recognition sound that causes the medical worker to recognize that the function of the instrument 12 has been estimated or that the estimation has been corrected is output at the time of estimation or correction. The recognition sound may be a beep sound or a certain message. For example, messages such as “ . . . has been registered in the patient ID . . . ” and “ . . . has been released in the patient ID . . . ” may be output.

The recognition sound may be output from the instrument 12 or may be output from the output unit 107 (FIG. 4) on the server 3 side.

In this manner, grouping of the cameras 11 is performed, what the instrument 12 imaged by the camera 11 is sensing (what is being monitored) is estimated, and in a case where the camera 11 and the instrument 12 are registered in association with the patient ID, the decision processing of deciding the analysis method and the data storage method corresponding to the sensing starts.

### <Storage Method Decision Processing>

In step S13, the camera 11 starts imaging. In step S14, the camera 11 supplies (transmits) the data of the imaged video to the server 3 in real time.

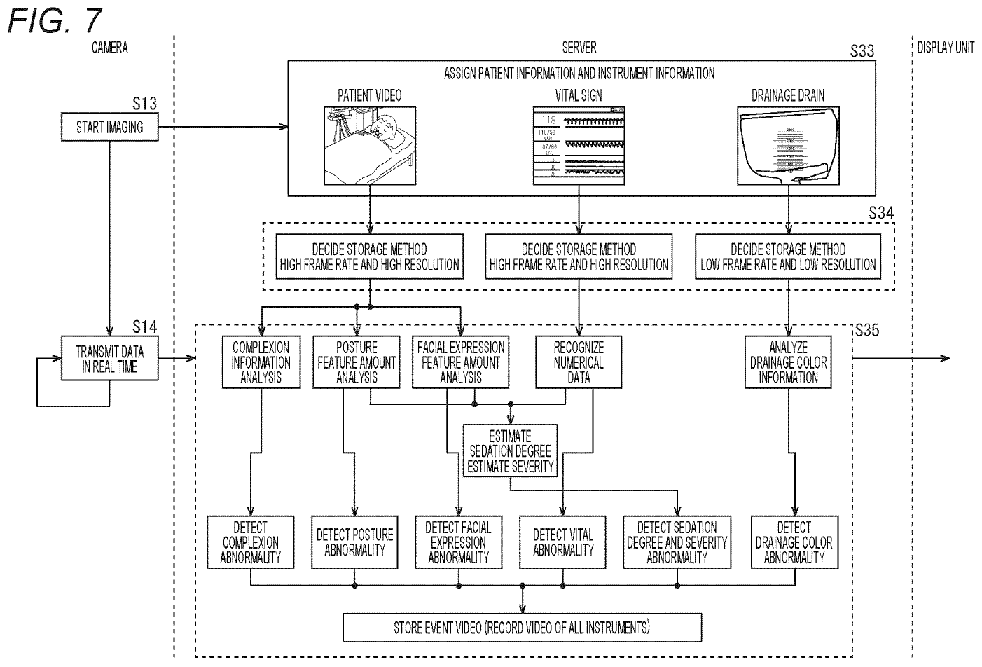

In a case where the camera 11 starts to image the instrument 12, the server 3 assigns the patient information and instrument information in step S33. In step S33, the patient information is assigned to the video data supplied from the camera 11. For example, with reference to FIG. 7, the video data from the camera 11 is imaging the patient, and the patient information indicating that the patient is, for example, the patient A is assigned to the image data from the camera 11 that is imaging the patient.

Referring to FIG. 7, the information indicating that the video data from the camera 11 is the video of the instrument 12 measuring the vital sign and is the vital sign of the patient A is assigned to the video data from the camera 11 imaging the instrument 12 measuring the vital sign.

Similarly, the information indicating that the video data from the camera 11 is a video of the instrument 12 imaging the drainage drain and measuring the amount of drainage and is the drainage drain connected to the patient A is assigned to the video data from the camera 11 imaging the drainage drain.

In a case where imaging by the camera 11 starts, the server 3 performs processing of associating the camera 11 with the information such as which patient's vital sign is being measured in the video imaged by the camera 11.

In step S34, the processing method decision unit 315 of the server 3 decides a storage method, analysis contents, a video layout, and the like. On the basis of the decision, in step S35, processing of analyzing and storing data transmitted from the camera 11 in real time is executed. The processing in steps S34 and S35 will be described with reference to FIG. 7.

In a case where all the data from the plurality of cameras 11 are stored in the storage unit 312, there is a possibility that an enormous storage capacity is required. Furthermore, in a case where all the data are stored, it may take time to search for necessary data, for example, a video or vital signs when the patient's condition has been changed. By storing only the data in a necessary time zone, the time required for storing and organizing the data is shortened, and the burden on the medical worker for management is reduced.

In order to reduce the data size of data to be stored, reduce the capacity for the storage in the storage unit 312, and shorten the time required for searching for necessary data, the storage method is optimized. As the optimization of the storage method (referred to as optimization 1), the data when it is determined that the patient's condition has been changed is stored in the storage unit 312.

For example, in a case where an event such as a sudden change in vital signs or a large movement of the patient occurs, data in the time zones before and after the time including the time is stored. By not storing data in a time zone in which no event has occurred, the data size of data to be stored can be reduced.

As the data in the time zone in which the event has occurred, video data of videos imaged by the plurality of cameras 11 and a plurality of analysis results obtained by analyzing the videos are stored. Since the condition of the patient can be analyzed using the information from the plurality of cameras 11, in a case where an abnormality occurs in a certain imaging target (instrument 12), the video data and analysis results of all the cameras 11 are stored.

As described above, in a case where an abnormality occurs in a certain imaging target (instrument 12), the video data and analysis results of all the cameras 11 are stored, and the videos from the plurality of cameras 11 cooperate with each other. Therefore, for example, in a case where there is an abnormality in the vital sign, it is possible to easily check what the patient's face is like.

As another optimization (referred to as optimization 2) of the storage method, the resolution is optimized to obtain a necessary and sufficient data size, and then the data is stored in the storage unit 312. For example, since a change in the face of the patient, for example, the complexion, the agonized facial expression, and the like need to be appropriately analyzed, the resolution of the video from the camera 11 that is imaging the face of the patient is set to be kept high.

For example, since the information desired to be obtained by observing the drainage drain is the color and amount of the drainage, the resolution of the video from the camera 11 imaging the drainage drain is set to be low.

As described above, by setting the resolution according to the observation target, the data size of the data to be stored can be set to an appropriate size, and the storage capacity for the storage can be reduced even in a case where the data from the plurality of cameras 11 is stored. By applying the optimization 1 and the optimization 2, it is possible to store the data in the time zone at the time of the occurrence of the event, at an appropriate resolution.

As another optimization (referred to as optimization 3) of the storage method, the frame rate is optimized to obtain a necessary and sufficient data size, and then the data is stored in the storage unit 312. For example, the frame rate of the camera 11 that is imaging the vital sign requiring real-time properties is set to be high. For example, the frame rate of the camera 11 imaging the drainage drain with a slow change speed is set to be low.

As described above, by setting the frame rate according to the observation target, the data size of the data to be stored can be set to an appropriate size, and the storage capacity for the storage can be reduced even in a case where the data from the plurality of cameras 11 is stored. By applying the optimization 1, the optimization 2, and the optimization 3, it is possible to store the data in the time zone at the time of the occurrence of the event, at an appropriate resolution and at an appropriate frame rate.

By performing such optimizations of the storage method and storing data, data at the time of occurrence of an event can be recorded, and labeling can be attached to the event. For example, in a case where the data is shared in an academic society or the like, it is possible to easily access a past video to which the labeling of the event is attached.

When the data is stored, not only the video data but also the analysis result, for example, the results of the severity estimation and the sedation degree estimation are stored, so that, for example, such a result can also be referred to and decided for the direction decision of the bed control of the ICU and the patient management method.

Referring to FIG. 7, it is decided that the patient video imaged by the camera 11 is stored at a high frame rate and with high resolution as the storage method. It is decided that the video of the vital sign imaged by the camera 11 is stored at a high frame rate and with high resolution as the storage method. It is decided that the video of the drainage drain imaged by the camera 11 is stored at a low frame rate and with low resolution as the storage method.

In this manner, an appropriate frame rate or resolution is set for each instrument 12, and data is stored at the set frame rate or with the set resolution.

For the decision of the analysis content, for example, the analysis content of performing the severity estimation of the patient is decided using the patient information and the vital sign read from the video is decided for the video from the camera 11 imaging the vital sign.

For example, the analysis content of analyzing the video from the camera 11 imaging the vital sign and the video from the camera 11 imaging the face and posture of the patient and of estimating the severity, the sedation degree, and the like of the patient is decided.

For example, in the case of the video from the camera 11 imaging the drainage drain, the analysis content of detecting the drain portion and performing color analysis such as color difference and blood detection is decided.

For example, in the case of the video from the camera 11 imaging the drainage drain for storing urine, the analysis content of detecting the drain portion and performing color analysis such as a color difference, concentration, and the like of urine is decided.

For example, in the case of the video from the camera 11 imaging the face of the patient, the analysis content of detecting the landmark of the face and analyzing the pigment in the same region of the face is decided.

These analysis methods can be configured to be additionally introduced as an analysis application. In this manner, the version of the application can be upgraded as an extended function, and a more appropriate analysis can be maintained.

The processing method decision unit 315 of the server 3 decides the analysis method suitable for the instrument 12 being imaged. In step S34, the server 3 also decides the video layout. The video layout will be described later with reference to screen examples (FIGS. 10, 11, and 12) displayed on the display unit 401.

In step S34, on the basis of the analysis method decided by the processing method decision unit, the analysis unit 316 analyzes the video (information from the instrument 12) from each camera 11. In step S36, the video layout processing unit 317 controls the display of the video and the analysis result to be displayed on the display unit 401 on the basis of the video layout decided by the processing method decision unit 315.

In step S51, the display unit 401 accepts an instruction from the medical worker who is viewing the displayed screen. The instruction from the medical worker is, for example, an instruction of a display mode for selecting information to be displayed, such as patient selection, analysis result selection, and video selection.

In a case of accepting an instruction issued by the medical worker operating the operation unit (input unit 106) while the medical worker viewing the screen displayed on the display unit 401, the instruction is supplied to the video layout processing unit 317. The video layout processing unit 317 controls the display of the display unit 401 so as to perform the display in the display mode based on the instruction. As a result, in step S52, a screen (UI) in the display mode instructed by the medical worker is displayed on the display unit 401.

Specific processing of such processing will be described again with reference to FIG. 7.

As the processing of step S35, the analysis unit 316 performs an analysis for generating complexion information, an analysis for extracting a posture feature amount, and an analysis for extracting a facial expression feature amount, using a patient video from the camera 11 imaging the patient.

The abnormality detection of the complexion is performed using the generated complexion information. The abnormality detection of the posture is performed using the generated posture feature amount. The abnormality detection of the facial expression is performed using the generated facial expression feature amount. In a case where at least one abnormality is detected, it is determined that an event has occurred, and all analysis results and video data are stored in the storage unit 312.

As the processing of step S35, the analysis unit 316 recognizes numerical data and acquires numerical values of the vital signs such as pulse, blood pressure, and body temperature, for example, by using the vital signs from the camera 11 imaging the instrument 12 measuring the vital signs. The abnormality detection of the vital is performed by determining whether or not the acquired numerical values of the vital signs are abnormal. In a case where the abnormality is detected, it is determined that an event has occurred, and all analysis results and video data are stored in the storage unit 312.

The sedation degree estimation and the severity estimation are performed using the numerical data of the posture feature amount, the facial expression feature amount, and the vital signs. According to the present technology, a plurality of instruments 12 (subjects including the patient) is imaged by a plurality of cameras 11, and the state of the patient can be estimated by comprehensively using the information from the plurality of instruments 12. Therefore, it is possible to perform a more accurate analysis than the analysis performed using the information from one instrument 12, and it is possible to more reliably detect the abnormality such as a sudden change in the state of the patient.

The abnormality detection is performed by using the estimated sedation degree estimation and severity, and in a case where the abnormality is detected, it is determined that an event has occurred, and all analysis results and video data are stored in the storage unit 312.

As the processing of step S35, the analysis unit 316 analyzes (acquires) the color information of the drainage using the video of the drainage drain from the camera 11 imaging the instrument 12 imaging the drainage drain. The abnormality detection of the color of the drainage is performed using the acquired color information, and in a case where the abnormality is detected, it is determined that an event has occurred, and all analysis results and video data are stored in the storage unit 312.

In this manner, it is possible to estimate the state of the patient and detect the abnormality by acquiring and analyzing the video data obtained by the camera 11 imaging the information from the patient or the instrument 12 (subject) as the imaging target.

Among these analyses, the analysis of the posture feature amount and the analysis of the facial expression feature amount will be further described.

The face feature amount is extracted from the face image of the patient acquired from the predetermined camera 11. For example, a numerical value indicating the state of agony based on the facial expression of the patient is extracted from the face image as the face feature amount.

The patients admitted to the ICU often wear a ventilator. Since the ventilator hides a part of the face of the patient, in a case where a general-purpose facial expression detection technology is used to extract the face feature amount, the accuracy of facial expression detection may deteriorate.

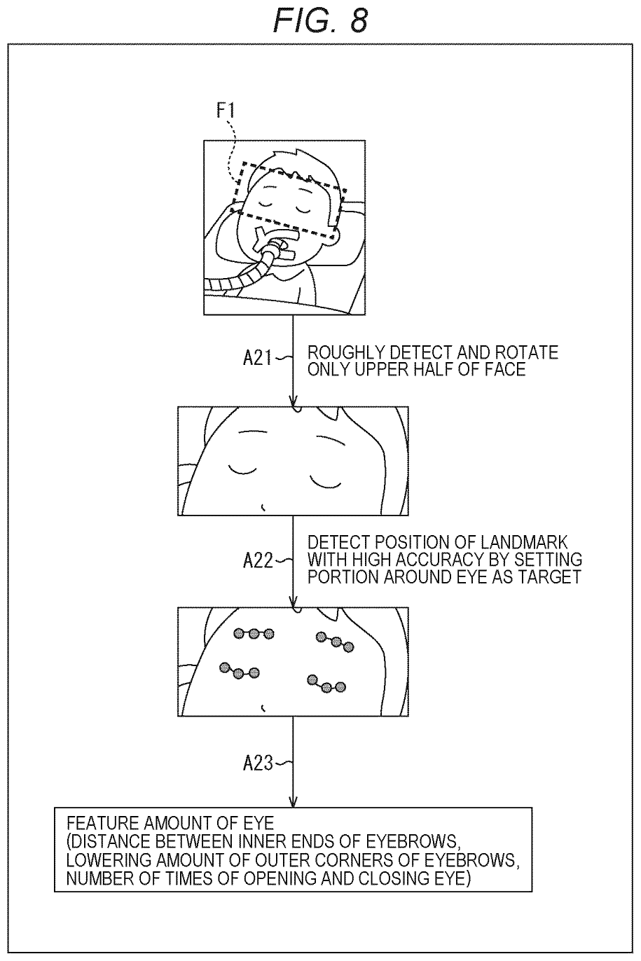

The analysis unit 316 of the server 3 performs facial expression recognition specialized for extraction of the feature amount around the patient's eye as the face feature amount. FIG. 8 is a diagram illustrating a flow of an extraction method of a feature amount around the eye.

As indicated by an arrow A21 in FIG. 8, the server 3 roughly detects a region showing the upper half of the patient's face from the face image. In the example of FIG. 8, as illustrated in a rectangular frame F1, a region around the eye surrounding from the nose to the forehead of the patient is detected as a region used for the feature amount extraction around the eyes.

The server 3 cuts out the region around the eyes from the face image to generate a partial image. After rotating the partial image around the eyes, the server 3 detects landmarks around the eyes from the image as indicated by an arrow A22. For example, at least one of the position of the edge of the eyelid, the center position of the eye (the center position of the iris), the position of the eyebrow, the position of the inner corner of the eye, the position of the outer corner of the eye, and the position of the ridge of the nose is detected as the position of the landmark around the eyes. The gray dots on the partial image around the eyes indicate the positions of the landmarks around the eyes.

By setting only the region around the eyes as a target of landmark detection, it is possible to detect the landmark with high accuracy without being affected by the ventilator.

As indicated by an arrow A23, the server 3 extracts feature amounts around the eyes such as a distance between the inner ends of the eyebrows, an opening state of the eyelids, the number of times of opening and closing the eyelids, a lowering amount of the outer corners of the eyes, and a direction of the eye lines, for example, on the basis of the positions of the landmarks around the eyes.

The feature amount around these eyes is a numerical value indicating the patient's agony, depression, vigor, and the like. Note that information indicating a relative positional relationship between landmarks around the eyes may be used as the feature amount around the eyes.

The server 3 can use the feature amount around the eyes extracted from the face image as the facial expression feature amount.

The server 3 extracts the posture feature amount from a whole-body image. For example, a numerical value indicating an excited state based on the spasm or movement of the patient's body is extracted from the whole-body image as the posture feature amount.

A patient admitted to the ICU may be covered with futon bedding. Since the futon conceals a part of the patient's body, in a case where a general-purpose skeleton estimation technique is used to extract the posture feature amount, the accuracy of skeleton estimation may deteriorate.

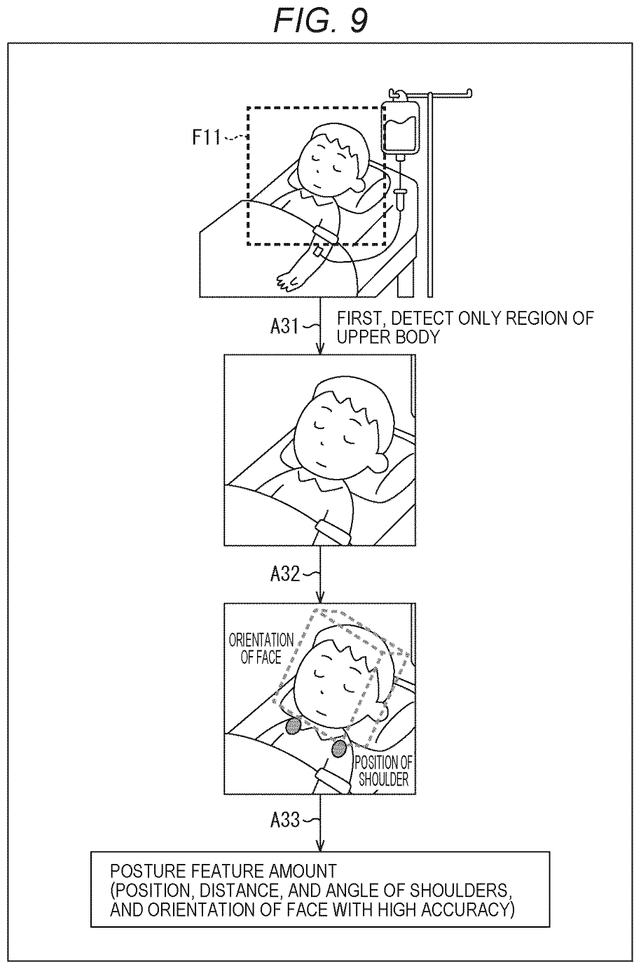

Therefore, the server 3 performs recognition specialized for extraction of the feature amounts of the face and the shoulder of the patient. FIG. 9 is a diagram illustrating a flow of an extraction method of a feature amount of the face and the shoulder.

As indicated by an arrow A31 in FIG. 9, the server 3 roughly detects a region showing the upper body of the patient from the whole-body image. In the example of FIG. 9, a region surrounded by a rectangular frame F11 is detected as a region used for extraction of the feature amounts of the face and the shoulder.

The server 3 cuts out a region of the upper body from the whole-body image to generate a partial image. After generating the partial image of the upper body, the server 3 detects the orientation of the face and the position of the shoulder from the partial image of the upper body as indicated by an arrow A32. A dashed square on the partial image of the upper body indicates the orientation of the face of the patient. Furthermore, two gray ellipses indicate the position of the shoulder.

By setting only the region of the upper body as the target of the detection of the position of the shoulder, the position of the shoulder can be detected with high accuracy without being affected by the futon.

As indicated by an arrow A33, the server 3 extracts the position of the shoulder, the distance between the shoulders, the angle between the shoulders, the direction of the face, and the like as the posture feature amount. Specifically, on the basis of the position of the shoulder and the orientation of the face, numerical values such as an angle at which the body rotates leftward with reference to the supine state, an angle at which the face tilts with reference to the shoulder, and an angle at which the right shoulder rises with reference to the left shoulder are obtained as the posture feature amount.

The face feature amount and the posture feature amount extracted from the video as described above are used for the analysis in the subsequent stage. For example, by performing multivariate analysis using time-series data of the feature amounts, the abnormality such as a sudden change in the patient's condition is detected.

Referring again to FIG. 7, the estimation of the sedation degree and the severity is performed using the numerical data of the posture feature amount, the facial expression feature amount, and the vital signs. The posture feature amount and the facial expression feature amount are obtained as described above.

For example, it is possible to determine whether or not the patient is moving greatly using the posture feature amount, and in a case where the patient is moving greatly, it is possible to output an analysis result indicating that the patient is suffering. For example, it is possible to determine whether or not the facial expression indicates that the patient is suffering using the facial expression feature amount, and in a case where it is determined that the facial expression indicates that the patient is suffering, it is possible to output an analysis result indicating that the patient is suffering. It is possible to determine whether or not the patient is suffering and whether or not the condition is worsening using the numerical data of the vital signs, and in a case where it is determined that the condition is worsening, it is possible to output an analysis result indicating that the condition is worsening.

These analysis results can be integrated to determine whether or not the patient is in a calm state or in a severe state. As described above, it is possible to estimate the sedation degree and the severity only with one analysis result, but it is possible to estimate the more accurate sedation degree and severity by using a plurality of analysis results.

In this manner, the patient's condition is determined by analyzing the video data obtained from the plurality of cameras 11. The determination result and the like are presented on a screen with a layout that is easy for the medical worker to understand. The screen will be described.

### <Video Layout>

A screen as illustrated in FIG. 10 is displayed on the display unit 401 as the output unit 107 of the server 3. The screen illustrated in FIG. 10 is a screen looking down the entire room, and illustrates a case where there are four patients A, B, C, and D in the room. The screen illustrated in FIG. 10 may be, for example, a video imaged from an indoor camera capable of imaging the entire room, or may be a drawing imitating a hospital room.

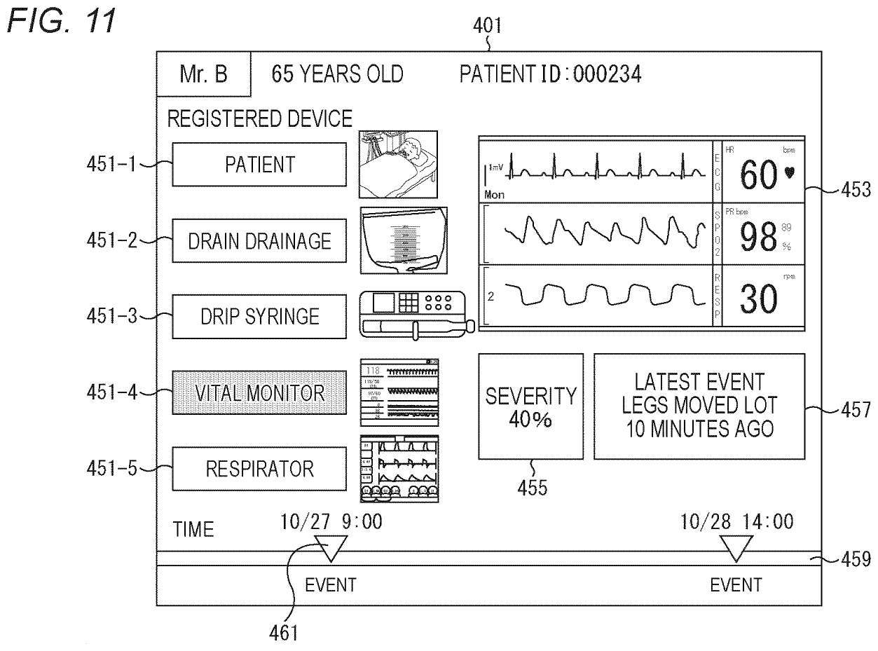

Buttons 421-1 to 421-4 with letters of patient A, patient B, patient C, and patient D are displayed. For example, in a case where a predetermined operation such as clicking is performed on the button 421-2 with a description such as the patient B, the display of the display unit 401 is switched to a screen for displaying information associated with information regarding the patient B as illustrated in FIG. 11.

On the upper side of the screen, there are display of a name such as Mr. B, display of an age such as 65 years old, and display of a patient ID, and display is made to cause the medical worker to recognize that the screen is a screen of the patient B. On the left side of the screen, a button 451-1 with a description such as a patient, a button 451-2 with a description such as drain drainage, a button 451-3 with a description such as a drip syringe, a button 451-4 with a description such as a vital monitor, and a button 451-5 with a description such as a respiratory are displayed as the registered device.

The registered device indicates the instrument 12 registered in step S32 (FIG. 6). At the registered device, corresponding buttons 451 are displayed by the number of registered instruments 12. Since the type and the number of installed instruments 12 are different depending on the patient, the screen as illustrated in FIG. 11 is a screen optimized for each patient.

A layout for displaying such an optimized screen is set as the video layout by the processing method decision unit 315 in step S34 (FIG. 6). On the basis of the video layout decided by the processing method decision unit 315, the video layout processing unit 317 controls the display of the display unit 401, and thereby the display of the screens illustrated in FIGS. 10 to 12 is controlled.

On the right side of each button 451 in the drawing, a video or an image from the camera 11 that is imaging the registered device (instrument 12) is displayed. A video may be displayed, or one captured frame may be displayed as an image. Instead of a video or an image based on the video data supplied from the camera 11, a drawing or a picture imitating the registered device may be used.

A monitoring image display region 453 in which an image (video) corresponding to the selected button 451 is displayed is provided in the upper part on the right side in the drawing. The example illustrated in FIG. 11 is an example of a case where the button 451-4, for which the registered device is a vital monitor, is operated, and a monitoring screen of vital signs is displayed in the monitoring image display region 453. In the monitoring image of the vital signs, the vital signs of the ICU patient measured by a vital sensor are displayed, and for example, an electrocardiogram (ECG), saturation of percutaneous oxygen (SpO2), and respiratory (RESP) are displayed.

Although not illustrated, in a case where the button 451-1 with a description such as a patient is operated, a video from the camera 11 that is imaging the patient B is displayed in the monitoring image display region 453. Similarly, also in a case where another button 451 is operated, a video from the corresponding camera 11 is displayed. Therefore, the medical worker can monitor the status of the patient B even in a case where the medical worker is in a place different from the hospital room where the patient B is.

A severity display region 455 is provided in the lower left part of the monitoring image display region 453 in the drawing. In the severity display region 455, in the example illustrated in FIG. 11, the severity of the patient B is displayed, and it is displayed that the severity is 40%.

An event display region 457 is provided in the lower right part of the monitoring image display region 453 in the drawing. In the event display region 457, for example, information associated with information regarding the most recently occurred event is displayed. In the example illustrated in FIG. 11, display of “LATEST EVENT, 10 MINUTES AGO, LEGS MOVED LOT” is made.

On the lower side of the screen, a bar 459 that displays the time when the event has occurred is displayed. A character string such as an event, a mark 461, and an occurrence time are displayed at a position on the bar 459 corresponding to the time when the event has occurred. The mark 461 is a button, and is configured such that in a case where the mark 461 is operated, a video at the time of occurrence of the event can be browsed in another window. A character string such as an event may function as a button.

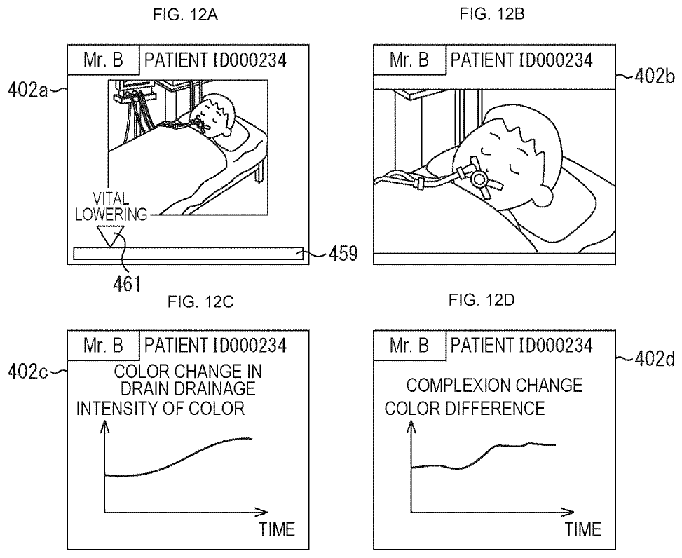

In a case where the mark 461 is operated, a window 402a as illustrated in A of FIG. 12 is displayed on the display unit 401. In the window 402a illustrated in A of FIG. 12, a video of the patient B at the time of occurrence of the event and a display indicating that the lowering of the vital has occurred as the event are displayed. The bar 459 and the mark 461 are also displayed in the window 402a.

For example, a mechanism may be provided in which a video of the patient B at a desired time before and after the occurrence of the event is displayed by sliding the mark 461 on the bar 459. In a case where the mark 461 is operated, in the example illustrated in A of FIG. 12, instead of the video of the patient B, the vital sign at the time of occurrence of the event may be displayed in the region where the video of the patient B is displayed.

A face image of the patient B is displayed in a window 402b illustrated in B of FIG. 12. Such a window 402b is, for example, a window that is opened by a predetermined operation such as clicking on the monitoring image display region 453 in a state where the button 451-1 with a description such as a patient is selected on the screen illustrated in FIG. 11 and in a state where the video (image) of the patient B is displayed in the monitoring image display region 453.

In a case where a predetermined operation, for example, clicking is performed on the monitoring image display region 453, a window for displaying the monitoring image displayed in the monitoring image display region 453 in an enlarged manner can be opened.

A window 402c illustrated in C of FIG. 12 is an example of a window that is opened when an image of the drain drainage is displayed in the monitoring image display region 453 in a state where the button 451-2 with a description such as drain drainage is selected. An image obtained by enlarging the drain drainage may be displayed on the window 402c, but a graph as illustrated in C of FIG. 12 may be displayed.

In the window 402c illustrated in C of FIG. 12, a graph in which a color change of the drain drainage is graphed is displayed. The horizontal axis of the graph displayed in the window 402c is time, and the vertical axis thereof is intensity of color. As described above, in a case where the monitoring image display region 453 is clicked, the image of the sensing target displayed in the monitoring image display region 453 may be displayed, or the result of analysis from the video data of the sensing target may be displayed.

The example illustrated in D of FIG. 12 is another example in which a result of analysis from the video data of the sensing target is displayed in the window. The example illustrated in D of FIG. 12 is an example of a window that is opened when a face image of the patient B is displayed in the monitoring image display region 453 in a state where the button 451-1 with a description such as a patient is selected.

In a window 402d illustrated in D of FIG. 12, a graph in which a complexion change of the patient is graphed is displayed. The graph illustrated in D of FIG. 12 is a graph in a case where the horizontal axis is time and the vertical axis is the color difference of the complexion. In this way, in a case where the monitoring image display region 453 in which the face image is displayed is operated, information acquired by analyzing the face image (video data of the face), in this case, information of the complexion may be displayed in the window.

As described above, according to the present technology, information obtained from the plurality of cameras 11 can be displayed as a user interface (UI) that is easy for the medical worker to see.

For example, in the ICU, it is assumed that there are many movements of the bed and the instruments, and the connected instruments are different for each patient. Therefore, if all the work of associating the patient, the instruments 12, and the sensing device (camera 11) is manually performed, the burden on the medical worker is large. However, according to the present technology, such association work can be easily performed in a short time without human error.

The present technology can also be applied to an ICU that is called Tele-ICU or the like and is supported by a medical worker at a remote location. Since the video obtained by imaging the instrument 12 of interest can be seen on the server 3 side, that is, at a place away from the hospital room, the medical worker can check necessary information as necessary without going to the bedside of the patient.

The present technology can also be applied to round-visit called remote multi-job round-visit or the like. For infectious disease countermeasures and the like, medical staffs may not gather around the patient, and multi-job round-visit may be performed remotely. At the time of such remote multi-job round-visit, in the site to which the present technology is applied, multi-view imaging is performed, and thus, it is possible to select or zoom a place of interest for each medical staff.

The present technology can also be applied to a check called a remote double check or the like. For example, there is a case where double check is performed so that an incident does not occur due to setting of a drip, setting of a ventilator, or the like, but at the time of this check, even if the medical worker is not at the site, the check can be performed using the video of a remote destination.

Note that the effects described in the present specification are merely examples and are not limited, and there may be other effects.

Note that embodiments of the present technology are not limited to the above-described embodiments, and various changes can be made without departing from the scope of the present technology.

Note that the present technology can also have the following configuration.

(1) An information processing device including:
an acquisition unit that acquires at least video data from an imaging device that images a patient and video data from an imaging device that images an instrument monitoring the patient; and
a detection unit that detects an abnormality of a condition of the patient by analyzing the video data acquired by the acquisition unit.
   (2)

The information processing device according to (1),
in which a plurality of the imaging devices that images a plurality of the instruments monitoring the patient is grouped by reading a patient ID assigned to the patient using the imaging device.
   (3)

The information processing device according to (2),
in which reading the patient ID is performed by the imaging device set as a master unit among the plurality of imaging devices, and the imaging device set as the master unit and the imaging device set as a slave unit are paired.
   (4)

The information processing device according to (3),
in which, by the imaging device set as the master unit, a face of the patient is imaged, the imaged face of the patient is collated with a face of the patient stored in a database, and the imaging device and the patient are associated with each other.
   (5)

The information processing device according to (1),
in which the imaging devices of which a distance is close to each other are grouped.
   (6)

The information processing device according to any one of (1) to (5),
in which a monitoring target of the instrument imaged by the imaging device is estimated.
   (7)

The information processing device according to (6),
in which an analysis method suitable for the estimated target is set, and the video data is analyzed on a basis of the set analysis method.
   (8)

The information processing device according to (6) or (7),
in which a storage method of data suitable for the estimated target is set, and the video data is stored on a basis of the set storage method.
   (9)

The information processing device according to (8),
in which the storage method is to set a frame rate and resolution suitable for the target.
   (10)

The information processing device according to (8),
in which in a case where the abnormality of the condition of the patient is detected by the detection unit, the video data is stored on the basis of the storage method.
   (11)

The information processing device according to (8),
in which in a case where the abnormality of the condition of the patient is detected by the detection unit, video data imaged by the grouped imaging devices and an analysis result obtained by analyzing the video data are stored on the basis of the storage method.
   (12)

The information processing device according to any one of (1) to (11),
in which at least one of complexion, a posture, and a facial expression of the patient is analyzed using video data from the imaging device that images the patient.
   (13)

The information processing device according to any one of (1) to (12),
in which a numerical value of vital signs is recognized using video data from the imaging device that images the instrument measuring the vital signs of the patient.
   (14)

The information processing device according to any one of (1) to (13),
in which a color of drainage is analyzed using video data from the imaging device that images a drainage drain.
   (15)

The information processing device according to any one of (1) to (14),
in which the condition of the patient is estimated using a plurality of analysis results obtained by analyzing video data acquired from each of a plurality of the imaging devices.
   (16)

An information processing method including:
by an information processing device,
acquiring at least video data from an imaging device that images a patient and video data from an imaging device that images an instrument monitoring the patient; and
detecting an abnormality of a condition of the patient by analyzing the acquired video data.
   (17)

A program for causing a computer that controls an information processing device to execute processing including steps of:
acquiring at least video data from an imaging device that images a patient and video data from an imaging device that images an instrument monitoring the patient; and
detecting an abnormality of a condition of the patient by analyzing the acquired video data.

### REFERENCE SIGNS LIST

1 Information processing system
2 Network
3 Server
11 Camera
12 Instrument
31 Arm
33 Mirror
101 CPU
102 ROM
103 RAM
104 Bus
105 Input/output interface
106 Input unit
107 Output unit
108 Storage unit
109 Communication unit
110 Drive
111 Removable medium
301 Information acquisition unit
302 Information processing unit
311 Grouping processing unit
312 Storage unit
313 Estimation processing unit
314 Registration unit
315 Processing method decision unit
316 Analysis unit
317 Video layout processing unit
321 Storage unit
401 Display unit
402 Window
421 Button
453 Monitoring image display region
455 Severity display region
457 Event display region
459 Bar
461 Mark

1. An information processing device comprising:
an acquisition unit that acquires at least video data from an imaging device that images a patient and video data from an imaging device that images an instrument monitoring the patient; and
a detection unit that detects an abnormality of a condition of the patient by analyzing the video data acquired by the acquisition unit.

2. The information processing device according to claim 1,
wherein a plurality of the imaging devices that images a plurality of the instruments monitoring the patient is grouped by reading a patient ID assigned to the patient using the imaging device.

3. The information processing device according to claim 2,
wherein reading the patient ID is performed by the imaging device set as a master unit among the plurality of imaging devices, and the imaging device set as the master unit and the imaging device set as a slave unit are paired.

4. The information processing device according to claim 3,
wherein, by the imaging device set as the master unit, a face of the patient is imaged, the imaged face of the patient is collated with a face of the patient stored in a database, and the imaging device and the patient are associated with each other.

5. The information processing device according to claim 1,
wherein the imaging devices of which a distance is close to each other are grouped.

6. The information processing device according to claim 1,
wherein a monitoring target of the instrument imaged by the imaging device is estimated.

7. The information processing device according to claim 6,
wherein an analysis method suitable for the estimated target is set, and the video data is analyzed on a basis of the set analysis method.

8. The information processing device according to claim 6,
wherein a storage method of data suitable for the estimated target is set, and the video data is stored on a basis of the set storage method.

9. The information processing device according to claim 8,
wherein the storage method is to set a frame rate and resolution suitable for the target.

10. The information processing device according to claim 8,
wherein in a case where the abnormality of the condition of the patient is detected by the detection unit, the video data is stored on the basis of the storage method.

11. The information processing device according to claim 8,
wherein in a case where the abnormality of the condition of the patient is detected by the detection unit, video data imaged by the grouped imaging devices and an analysis result obtained by analyzing the video data are stored on the basis of the storage method.

12. The information processing device according to claim 1,
wherein at least one of complexion, a posture, and a facial expression of the patient is analyzed using video data from the imaging device that images the patient.

13. The information processing device according to claim 1,
wherein a numerical value of vital signs is recognized using video data from the imaging device that images the instrument measuring the vital signs of the patient.

14. The information processing device according to claim 1,
wherein a color of drainage is analyzed using video data from the imaging device that images a drainage drain.

15. The information processing device according to claim 1,
wherein the condition of the patient is estimated using a plurality of analysis results obtained by analyzing video data acquired from each of a plurality of the imaging devices.

16. An information processing method comprising:
by an information processing device,
acquiring at least video data from an imaging device that images a patient and video data from an imaging device that images an instrument monitoring the patient; and
detecting an abnormality of a condition of the patient by analyzing the acquired video data.

17. A program for causing a computer that controls an information processing device to execute processing including steps of:
acquiring at least video data from an imaging device that images a patient and video data from an imaging device that images an instrument monitoring the patient; and
detecting an abnormality of a condition of the patient by analyzing the acquired video data.
